(19) United States
(12) Patent Application Publication
(10) Pub. No.: US 2008/0177079 A1
(43) Pub. Date: Jul. 24, 2008

(54) NOVEL PROCESS FOR THE PREPARATION OF (2R)-2-DEOXY-2-FLUORO-2-METHYL-D-ERYTHRO-PENTONO-GAMMA-LACTONE AND (2S)-2-DEOXY-2-FLUORO-2-METHYL-D-ERYTHRO-PENTONO-GAMMA-LACTONE

(76) Inventors: Miall Cedilote, Florence, SC (US); Thomas Cleary, Florence, SC (US); Pingsheng Zhang, Florence, SC (US)

Correspondence Address: HOFFMANN-LA ROCHE INC.;PATENT LAW DEPARTMENT, 340 KINGSLAND STREET, NUTLEY, NJ, 07110, US

(21) Appl. No.: 12/014,124

(22) Filed: Jan. 15, 2008

Related U.S. Application Data
(60) Provisional application No. 60/881,970, filed on Jan. 23, 2007.

(51) Int. Cl. C07D 263/62; C07D 207/00

(52) U.S. Cl. 548/221; 548/530

(57) ABSTRACT

The present invention provides novel methods for preparing compounds (2R)-2-deoxy-2-fluoro-2-methyl-D-erythro-pentono-γ-lactone (11) and (2S)-2-deoxy-2-fluoro-2-methyl-D-erythro-pentono-γ-lactone (12), which are intermediates for preparing a key intermediate 3,5-di-O-acyl-2-fluoro-2-C-methyl-D-ribono-γ-lactone (B), for the preparation of 1-(2-deoxy-2-fluoro-2-C-methyl-β-D-ribofuranosyl)cytosine (A), which is a potent and selective anti-hepatitis C virus agent.

### PRIORITY TO RELATED APPLICATIONS

This application claims the benefit of U.S. Provisional Application No. 60/881,970, filed Jan. 23, 2007, which is hereby incorporated by reference in its entirety.

### FIELD OF THE INVENTION

The present invention provides novel methods for preparing compounds (2R)-2-deoxy-2-fluoro-2-methyl-D-erythro-pentono-γ-lactone (11) and (2S)-2-deoxy-2-fluoro-2-methyl-D-erythro-pentono-γ-lactone (12), which are intermediates for preparing a key intermediate 3,5-di-O-acyl-2-fluoro-2-C-methyl-D-ribono-γ-lactone (B), for the preparation of 1-(2-deoxy-2-fluoro-2-C-methyl-β-D-ribofuranosyl)cytosine (A), which is a potent and selective anti-hepatitis C virus agent.

### BACKGROUND OF THE INVENTION

Hepatitis C virus (HCV) infection is a major health concern that leads to chronic liver disease in a substantial number of patients. This viral disease is transmitted sexually and parenterally by contaminated blood, blood products, and contaminated needles. Current treatments for HCV infection are limited to immunotherapy with interferon-α, either alone, or in combination with ribavirin [1-(β-D-ribofuranosyl)-1H-1,2,4-triazole-3-carboxamide).

The HCV virion is a small, enveloped positive-strand RNA virus in the Flaviviridae family. The genome contains a single open reading frame encoding a polyprotein of over 3,000 amino acids, which is cleaved to generate the mature structural and non-structural viral proteins. The single open reading frame is flanked by 5′ and 3′ non-translated regions of a few hundred nucleotides in length, which are important for RNA translation and replication. The translated polyprotein contains the structural core and envelope proteins (E1, E2, p7) at the N-terminus, followed by the nonstructural proteins (NS2, NS3, NS4A, NS4B, NS5A, and NS5B). The mature structural proteins are generated via cleavage by the host signal peptidase. The junction between NS2 and NS3 is autocatalytically cleaved by the NS2/NS3 protease, while the remaining four junctions are cleaved by the N-terminal serine protease domain of NS3 complexed with NS4A. The NS3 protein also contains the NTP-dependent helicase activity, which unwinds duplex RNA during replication. The NS5B protein possesses RNA-dependent RNA polymerase activity, which is essential for viral replication. Unlike Hepatitis B virus (HBV) or Human Immunodeficiency Virus (HIV), no DNA is involved in the replication of HCV.

United States published patent application no. 2005-0009737 discloses that 1-(2-deoxy-2-fluoro-2-C-methyl-β-D-ribofuranosyl)cytosine (A) is a potent and selective anti-HCV agent. The synthetic procedures for preparing compound A are inefficient with overall yields at or below 4%.

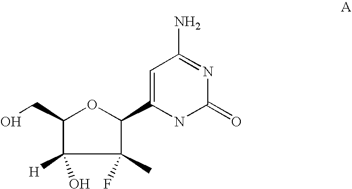

A key intermediate for preparing compound A is 3,5-di-O-acyl-2-fluoro-2-C-methyl-D-ribono-γ-lactone (B).

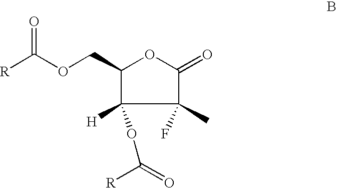

A number of synthetic routes for preparing intermediate B have been disclosed in PCT/US2005/025916, but these synthetic routes have the shortcomings of high manufacturing costs and technical difficulties for commercial scale manufacturing. The use of heavy load of asymmetric dihydroxlyation catalyst (AD-mix-β), fluorinating agent diethylaminosulfur trifluoride, and the Wittig reagent, etc., are the major cost drivers. The use of highly toxic reagents, such as AD-mix-β, highly reactive reagent such as diethylaminosulfur trifluoride, and chromatographic isolation of intermediates, etc. contribute to scale up difficulties.

Accordingly, novel and cost effective methods for preparing compounds, which are intermediates for preparing key intermediate B are required.

### SUMMARY OF THE INVENTION

The present invention provides a method for preparing compound 4 of the formula:

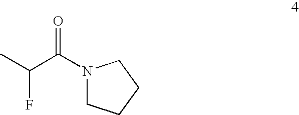

which comprises:

(a) converting 2-fluoropropionic acid to 2-fluoropropionyl chloride in a non-reactive solvent; and

(b) adding pyrrolidine to the mixture in step (a) to provide compound 4.

The present invention also provides a method for preparing compound 5 of the formula:

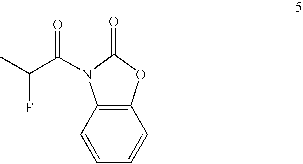

which comprises:

(a) adding a catalytic amount of a hypernucleophilic acylation catalyst to 2-fluoropropionic acid and 2-benzoxazolinone in a non-reactive solvent; and

(b) adding a dehydrating agent to the mixture in step (a) with stirring; and

(c) filtering the mixture in step (b) and concentrating the filtrate to provide compound 5.

The present invention further provides a method for preparing a mixture of compounds of the formula 11 and 12:

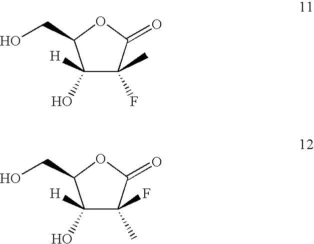

which comprises:

(a) admixing a non-nucleophilic base and 1-(2-fluoro-1-oxopropyl)pyrrolidine in a non-reactive solvent;

(b) admixing D-glyceraldehyde, 1,2-acetonide to the mixture from step (a);

(c) neutralizing the mixture from step (b) and partitioning the resulting mixture between a volatile organic solvent and water and concentrating the volatile organic solution to provide an intermediate;

(d) hydrolyzing the intermediate from step (c); and

(e) concentrating the mixture from step (d) to provide a residue and partitioning the residue between water and a volatile organic solvent and concentrating the water phase to provide a residue, which is a mixture of 11 and 12.

The present invention still further provides a method for preparing a mixture of compounds of the formula 11 and 12:

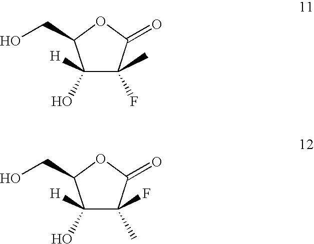

which comprises:

(a) admixing a non-nucleophilic base and S-phenyl-2-fluoropropanethioate in a non-reactive solvent;

(b) admixing D-glyceraldehyde, 1,2-acetonide to the mixture from step (a);

(c) neutralizing the mixture from step (b) and partitioning the resulting mixture between a volatile organic solvent and water and concentrating the volatile organic solution to provide an intermediate;

(d) hydrolyzing the intermediate from step (c); and

(e) concentrating the mixture from step (d) to provide a residue and partitioning the residue between water and a volatile organic solvent and concentrating the water phase to provide a residue, which is a mixture of 11 and 12.

The present invention still further provides a method for preparing a mixture of compounds of the formula 11 and 12:

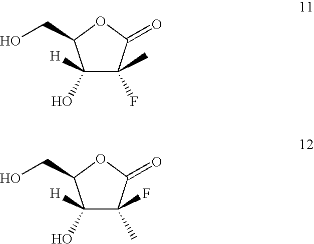

which comprises:

(a) admixing a non-nucleophilic base, S-phenyl-2-fluoropropanethioate, and chlorotriisopropoxytitanium (IV) in a non-reactive solvent;

(b) admixing D-glyceraldehyde, 1,2-acetonide to the mixture from step (a);

(c) neutralizing the mixture from step (b) and partitioning the resulting mixture between a volatile organic solvent and water and concentrating the volatile organic solution to provide an intermediate;

(d) hydrolyzing the intermediate from step (c); and

(e) concentrating the mixture from step (d) to provide a residue and partitioning the residue between water and a volatile organic solvent and concentrating the water phase to provide a residue, which is a mixture of 11 and 12.

The present invention still further provides a method for preparing a mixture of compounds of the formula 11 and 12:

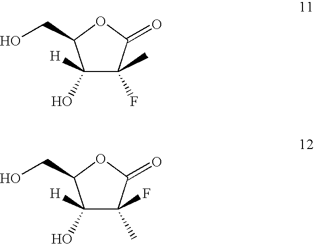

which comprises:

(a) admixing 3-(2-fluoro-1-oxopropyl)-2(3H)-benzoxazolone to a non-reactive solvent;

(b) admixing titanium (IV) chloride and a tertiary amine base to the mixture from step (a);

(c) admixing D-glyceraldehyde, 1,2-acetonide to the mixture from step (b);

(d) neutralizing the mixture from step (c) and partitioning the resulting mixture between a volatile organic solvent and water and concentrating the volatile organic solution to provide an intermediate;

(e) hydrolyzing the intermediate from step (d); and

(f) concentrating the mixture from step (e) to provide a residue and partitioning the residue between water and a volatile organic solvent and concentrating the water phase to provide a residue, which is a mixture of 11 and 12.

The present invention still further provides novel compounds 4 and 5 having the formulae set out below:

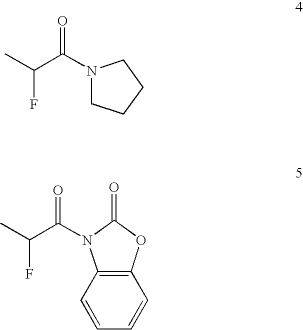

### DETAILED DESCRIPTION OF THE INVENTION

The present invention provides novel methods for preparing compounds (2R)-2-deoxy-2-fluoro-2-methyl-D-erythro-pentono-γ-lactone (11) and (2S)-2-deoxy-2-fluoro-2-methyl-D-erythro-pentono-γ-lactone (12), which are intermediates for preparing a key intermediate 3,5-di-O-acyl-2-fluoro-2-C-methyl-D-ribono-γ-lactone (B), for the preparation of 1-(2-deoxy-2-fluoro-2-C-methyl-β-D-ribofuranosyl)cytosine (A), which is a potent and selective anti-hepatitis C virus agent. The advantages of these new methods include 1) the use of less toxic and less expensive materials, and 2) fewer chemical transformations. The new methods result in a much more cost effective and operable manufacturing process

As used herein, the following terms have the given meanings:

The term “catalytic amount”, as used herein, refers to that amount of catalyst necessary to promote a chemical reaction. Although a catalyst undergoes no chemical change, it is often physically altered by the chemical reactants. The exact amount of catalyst necessary to promote a chemical reaction varies by the type of catalyst as well as the reactants employed and is readily determined by one skilled in the art.

The term “diastereomerically enriched” refers to a composition that comprises at least about 90%, and preferably about 95%, 98%, 99% or 100% of a single diastereomer of that composition.

The term “halogen” refers to chloro, bromo, iodo and fluoro, and is preferably chloro.

The term “non-reactive solvent” refers to a solvent that does not interfere chemically with the reaction. Preferred non-reactive solvents include dichloromethane, tetrahydrofuran, heptane, toluene, and ethylbenzene.

The term “pharmaceutically acceptable,” such as pharmaceutically acceptable carrier, excipient, etc., means pharmacologically acceptable and substantially non-toxic to the subject to which the particular compound is administered.

The term “pharmaceutically acceptable salt” refers to conventional acid-addition salts or base-addition salts that retain the biological effectiveness and properties of the compounds of the present invention and are formed from suitable non-toxic organic or inorganic acids or organic or inorganic bases. Sample acid-addition salts include those derived from inorganic acids such as hydrochloric acid, hydrobromic acid, hydroiodic acid, sulfuric acid, sulfamic acid, phosphoric acid and nitric acid, and those derived from organic acids such as p-toluenesulfonic acid, salicylic acid, methanesulfonic acid, oxalic acid, succinic acid, citric acid, malic acid, lactic acid, fumaric acid, and the like. Sample base-addition salts include those derived from ammonium, potassium, sodium, and quaternary ammonium hydroxides, such as for example, tetramethylammonium hydroxide. Chemical modification of a pharmaceutical compound (i.e., drug) into a salt is a technique well known to pharmaceutical chemists to obtain improved physical and chemical stability, hydroscopicity, and solubility of compounds. See, e.g., H. Ansel et. al., Pharmaceutical Dosage Forms and Drug Delivery Systems (6^{th} Ed. 1995) at pp. 196 and 1456-1457.

The term “prodrug” refers to compounds, which undergo transformation prior to exhibiting their pharmacological effects. The chemical modification of drugs to overcome pharmaceutical problems has also been termed “drug latentiation.” Drug latentiation is the chemical modification of a biologically active compound to form a new compound, which upon in vivo enzymatic attack will liberate the parent compound. The chemical alterations of the parent compound are such that the change in physicochemical properties will affect the absorption, distribution and enzymatic metabolism. The definition of drug latentiation has also been extended to include nonenzymatic regeneration of the parent compound. Regeneration takes place as a consequence of hydrolytic, dissociative, and other reactions not necessarily enzyme mediated. The terms prodrugs, latentiated drugs, and bio-reversible derivatives are used interchangeably. By inference, latentiation implies a time lag element or time component involved in regenerating the bioactive parent molecule in vivo. The term prodrug is general in that it includes latentiated drug derivatives as well as those substances, which are converted after administration to the actual substance, which combines with receptors. The term prodrug is a generic term for agents, which undergo biotransformation prior to exhibiting their pharmacological actions.

The term “protecting group” refers to a group that is added to an oxygen or nitrogen atom to prevent its further reaction. A wide variety of oxygen and nitrogen protecting groups are known to those skilled in the art of organic synthesis. Common protecting groups are disclosed in T. W. Greene and P. G. M. Wuts, “Protective groups in Organic Synthesis,” 3rd ed., John Wiley& Sons, 1999, which disclosure is incorporated herein by reference.

The term “volatile organic solvent” refers to a water-insoluble organic solvent having a boiling point below 130° C., preferably below 110° C., and more preferably below 100° C. Non-limiting examples of volatile organic solvents include methylene chloride, chloroform, carbon tetrachloride, diethyl ether, and the like.

The present invention provides novel methods for preparing compounds (2R)-2-deoxy-2-fluoro-2-methyl-D-erythro-pentono-γ-lactone (11) and (2S)-2-deoxy-2-fluoro-2-methyl-D-erythro-pentono-γ-lactone (12), which are intermediates for preparing a key intermediate 3,5-di-O-acyl-2-fluoro-2-C-methyl-D-ribono-γ-lactone (B), for the preparation of 1-(2-deoxy-2-fluoro-2-C-methyl-β-D-ribofuranosyl)cytosine (A), which is a potent and selective anti-hepatitis C virus agent. Compounds 11 and 12 were prepared from starting materials 1-(2-fluoro-1-oxopropyl)pyrrolidine (4), 3-(2-fluoro-1-oxopropyl)-2(3H)-benzoxazolone (5), and S-phenyl-2-fluoropropanethioate (7). S-phenyl-2-fluoropropanethioate (7) was prepared according to a literature method (Tetrahedron, 1996, 52 (1), 255). Compounds 4 and 5 were prepared according to Scheme 1, set out below.

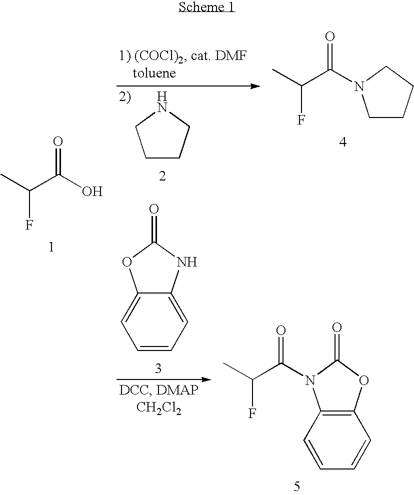

Compounds 11 and 12 were prepared according to Scheme 2, set out below.

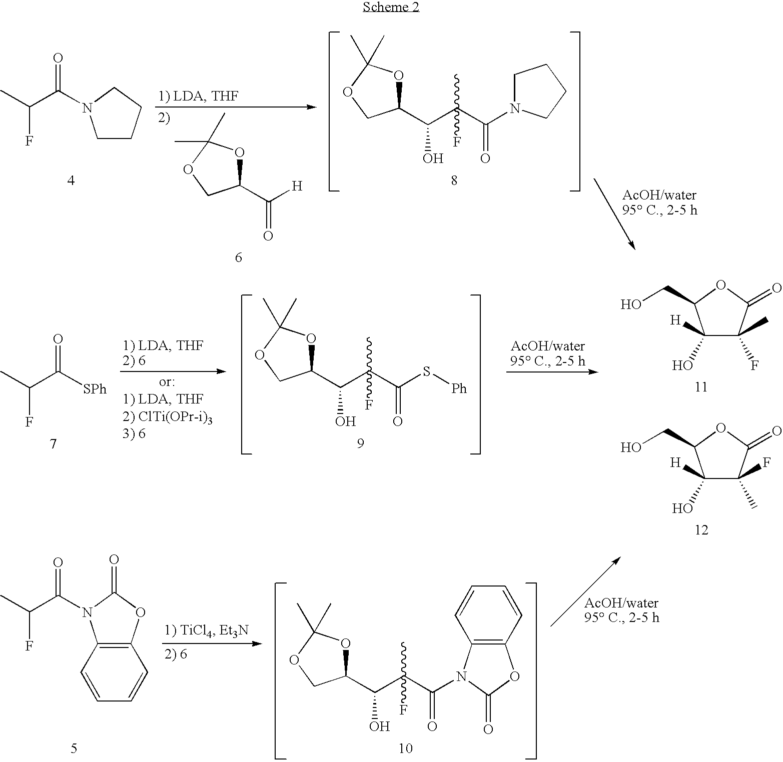

In a specific embodiment, the present invention provides a method for preparing compound 4 of the formula:

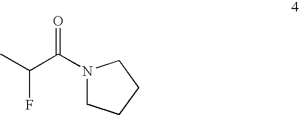

which comprises:

(a) converting 2-fluoropropionic acid to 2-fluoropropionyl chloride in a non-reactive solvent; and

(b) adding pyrrolidine to the mixture in step (a) to provide compound 4.

In step (a), converting 2-fluoropropionic acid to 2-fluoropropionyl chloride may be carried out using any reagent capable of converting carboxylic acids to their corresponding acyl halides. Preferably the reagent is an acyl halide that will convert compound (4) to its acyl halide. The term “acyl halide” refers to a group of the formula RC(O)X, wherein R is an alkyl or aromatic group and X is halogen. A preferred acyl halide is oxalyl chloride.

In step (a), the non-reactive solvent may be any solvent that does not chemically interfere with the reaction. A preferred non-reactive solvent is toluene.

The method may further comprise a catalytic amount of dimethylformamide (DMF, N,N-dimethylformamide) in step (a). Dimethylformamide is a clear liquid, miscible with water and a majority of organic solvents. Dimethylformamide is a polar (hydrophilic) aprotic solvent with a high boiling point and facilitates acyl chloride formation.

In a preferred embodiment of this invention, the method for preparing 4 comprises (a) adding oxalyl chloride to a solution of 2-fluoropropionic acid and a catalytic amount of dimethylformamide in anhydrous toluene; (b) stirring the mixture from step (a) at room temperature for about 2 h and at about 30° C. for about 1 h and then cooling the mixture to about −70° C.; (c) adding pyrrolidine to the mixture in step (b) and warming the mixture to ambient temperature; and (d) washing the mixture from step (c) with 5% HCl aqueous solution and 5% NaHCO₃ aqueous solution and concentrating the mixture to provide compound 4.

In another specific embodiment, the present invention provides a method for preparing compound 5 of the formula:

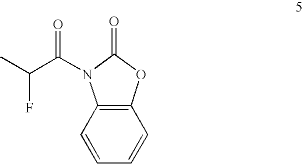

which comprises:

(a) adding a catalytic amount of a hypernucleophilic acylation catalyst to 2-fluoropropionic acid and 2-benzoxazolinone in a non-reactive solvent; and

(b) adding a dehydrating agent to the mixture in step (a) with stirring; and

(c) filtering the mixture in step (b) and concentrating the filtrate to provide compound 5.

In step (a), the hypernucleophilic acylation catalysts useful in the present invention are acylation catalysts. A catalyst is a compound used to promote a chemical reaction. Although a catalyst undergoes no chemical change, it is often physically altered by the chemical reactants. A hypernucleophilic acylation catalyst is a catalyst that accelerates the reaction rate of nucleophilic acylation substitution reactions. A nucleophilic substitution is the reaction of an electron pair donor (the nucleophile) with an electron pair acceptor (the electrophile). A nucleophilic acylation substitution reaction is a reaction where an acyl group is involved in the reaction. Non-limiting preferred hypernucleophilic acylation catalysts are 4-dimethylaminopyridine (DMAP) and dimethylformamide (DMF).

In step (b), the dehydrating agents useful in the present invention are agents that absorb water. Useful dehydrating agents are carbodiimides with functional groups consisting of the formula N═C═N. Compounds containing the carbodiimide functionality are dehydration agents and are often used to activate carboxylic acids towards amide or ester formation. The dehydrating agent in the present invention is preferably a carbodiimide selected from the group consisting of N,N′-dicyclohexylcarbodiimide, N,N′-diisopropylcarbodiimide, and 1-ethyl-3-(3-dimethylaminopropyl)carbodiimide hydrochloride. More preferably, the dehydrating agent is N,N′-dicyclohexylcarbodiimide.

In a preferred embodiment of this invention, the method for preparing 5 comprises (a) adding 2-fluoropropionic acid, 2-benzoxazolinone, and a catalytic amount of 4-dimethylaminopyridine in dichloromethane and cooling the mixture to about 0° C.; (b) adding N,N′-dicyclohexylcarbodiimide in dichloromethane to the mixture in step (a); (c) warming the mixture from step (b) to ambient temperature and stirring the mixture overnight; and (d) filtering the mixture from step (c) and concentrating the filtrate to provide compound 5.

In another specific embodiment, the present invention provides a method for preparing a mixture of compounds of the formula 11 and 12:

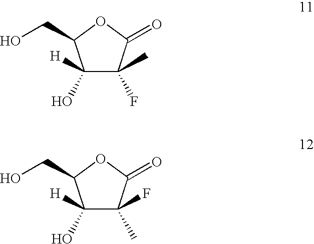

which comprises:

(a) admixing a non-nucleophilic base and 1-(2-fluoro-1-oxopropyl)pyrrolidine in a non-reactive solvent;

(b) admixing D-glyceraldehyde, 1,2-acetonide to the mixture from step (a);

(c) neutralizing the mixture from step (b) and partitioning the resulting mixture between a volatile organic solvent and water and concentrating the volatile organic solution to provide an intermediate;

(d) hydrolyzing the intermediate from step (c); and

(e) concentrating the mixture from step (d) to provide a residue and partitioning the residue between water and a volatile organic solvent and concentrating the water phase to provide a residue, which is a mixture of 11 and 12.

In step (a), any strong non-nucleophilic base may be used. A non-nucleophilic base is a sterically hindered base that has the ability to abstract an acidic hydrogen atom from a compound without otherwise chemically reacting with the compound, that is without displacing a functional group within the compound. Such displacements are generally referred to as nucleophilic substitutions. A nucleophilic substitution is the reaction of an electron pair donor (the nucleophile) with an electron pair acceptor (the electrophile). Tertiary amines are good examples of non-nucleophilic bases because they have the ability to abstract an acidic proton from a compound but because of their steric hindrance, they cannot otherwise react with the compound. Other non-limiting illustrative examples of non-nucleophilic bases include lithium diisopropylamide (LDA), lithium 2,2,6,6-tetramethylpiperdine (LTMP), lithium hexamethyldisilazide (LHMDS), and the like. Preferably, the non-nucleophilic base is lithium diisopropylamide.

In step (a), the reaction can be carried out in a non-reactive solvent, that is, a solvent that does not interfere with the reaction. Non-limiting illustrative examples include tetrahydrofuran (THF), 2-Me-THF, toluene, diethyl ether, tert-butyl methyl ether, and the like.

In a preferred embodiment of this invention, the method for preparing 11 and 12 comprises (a) admixing tetrahydrofuran and 1.8 M solution of lithium diisopropylamide in heptane/tetrahydrofuran/ethylbenzene; (b) cooling the mixture from step (a) to about −78° C. and admixing 1-(2-fluoro-1-oxopropyl)pyrrolidine in tetrahydrofuran; (c) stirring the mixture from step (b) for about 30 minutes and adding a solution of D-glyceraldehyde, 1,2-acetonide in tetrahydrofuran; (d) stirring the mixture from step (c) at about −75° C. for about 10 minutes and quenching the mixture with a solution of acetic acid in tetrahydrofuran; (e) partitioning the mixture from step (d) between dichloromethane and water and concentrating the dichloromethane phase to provide an intermediate; (f) mixing the intermediate from step (e) with acetic acid and water and stirring the mixture at about 90° C. overnight; and (g) concentrating the mixture from step (f) to provide a residue and partitioning the residue between water and tert-butyl methyl ether and concentrating the water phase to provide a residue which contains 63% of 11 and 37% of 12.

In another specific embodiment, the present invention provides a method for preparing a mixture of compounds of the formula 11 and 12:

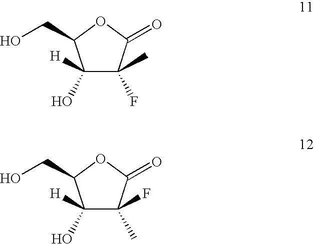

which comprises:

(a) admixing a non-nucleophilic base and S-phenyl-2-fluoropropanethioate in a non-reactive solvent;

(b) admixing D-glyceraldehyde, 1,2-acetonide to the mixture from step (a);

(c) neutralizing the mixture from step (b) and partitioning the resulting mixture between a volatile organic solvent and water and concentrating the volatile organic solution to provide an intermediate;

(d) hydrolyzing the intermediate from step (c); and

(e) concentrating the mixture from step (d) to provide a residue and partitioning the residue between water and a volatile organic solvent and concentrating the water phase to provide a residue, which is a mixture of 11 and 12.

The non-nucleophilic base is defined above.

In a preferred embodiment of this invention, the method for preparing a mixture of 11 and 12 comprises (a) admixing tetrahydrofuran and 1.8 M solution of lithium diisopropylamide in heptane/tetrahydrofuran/ethylbenzene; (b) cooling the mixture from step (a) to about −78° C. and admixing S-phenyl 2-fluoropropanethioate in tetrahydrofuran; (c) stirring the mixture from step (b) for about 30 minutes and adding a solution of D-glyceraldehyde, 1,2-acetonide in tetrahydrofuran; (d) stirring the mixture from step (c) at about −75° C. for about 10 minutes and quenching the mixture with a solution of acetic acid in tetrahydrofuran; (e) partitioning the mixture from step (d) between dichloromethane and water and concentrating the dichloromethane phase to provide an intermediate; (f) mixing the intermediate from step (e) with acetic acid and water and stirring the mixture at about 90° C. overnight; and (g) concentrating the mixture from step (f) to provide a residue and partitioning the residue between water and tert-butyl methyl ether and concentrating the water phase to provide a residue which contains 45% of 11 and 55% of 12.

In another specific embodiment, the present invention provides a method for preparing a mixture of compounds of the formula 11 and 12:

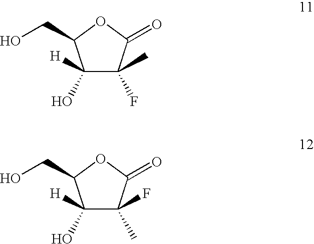

which comprises:

(a) admixing a non-nucleophilic base, S-phenyl-2-fluoropropanethioate, and chlorotriisopropoxytitanium (IV) in a non-reactive solvent;

(b) admixing D-glyceraldehyde, 1,2-acetonide to the mixture from step (a);

(c) neutralizing the mixture from step (b) and partitioning the resulting mixture between a volatile organic solvent and water and concentrating the volatile organic solution to provide an intermediate;

(d) hydrolyzing the intermediate from step (c); and

(e) concentrating the mixture from step (d) to provide a residue and partitioning the residue between water and a volatile organic solvent and concentrating the water phase to provide a residue, which is a mixture of 11 and 12.

The non-nucleophilic base is defined above.

In another preferred embodiment of this invention, the method for preparing a mixture of 11 and 12 comprises (a) admixing tetrahydrofuran and a 1.8 M solution of lithium diisopropylamide in heptane/tetrahydrofuran/ethylbenzene; (b) cooling the mixture from step (a) to about −78° C. and then admixing S-phenyl-2-fluoropropanethioate in tetrahydrofuran; (c) stirring the mixture from step (b) for about 1 h and then admixing chlorotriisopropoxytitanium (IV); (d) stirring the mixture from step (c) for about 1 h and adding a solution of D-glyceraldehyde, 1,2-acetonide in tetrahydrofuran; (e) stirring the mixture from step (d) at about −75° C. for about 10 minutes and quenching the mixture with a solution of acetic acid in tetrahydrofuran; (f) partitioning the mixture from step (e) between dichloromethane and water and concentrating the dichloromethane phase to provide an intermediate; (g) mixing the intermediate from step (f) with acetic acid and water and stirring the mixture at about 90° C. overnight; and (h) concentrating the mixture from step (g) to provide a residue and partitioning the residue between water and tert-butyl methyl ether and concentrating the water phase to provide a residue which contains 28% of 11 and 72% of 12.

In another specific embodiment, the present invention provides a method for preparing a mixture of compounds of the formula 11 and 12:

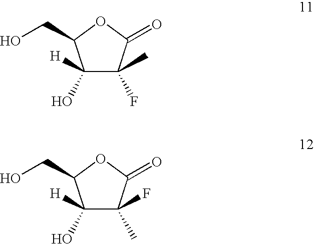

which comprises:

(a) admixing 3-(2-fluoro-1-oxopropyl)-2(3H)-benzoxazolone to a non-reactive solvent;

(b) admixing titanium (IV) chloride and a tertiary amine base to the mixture from step (a);

(c) admixing D-glyceraldehyde, 1,2-acetonide to the mixture from step (b);

(d) neutralizing the mixture from step (c) and partitioning the resulting mixture between a volatile organic solvent and water and concentrating the volatile organic solution to provide an intermediate;

(e) hydrolyzing the intermediate from step (d); and

(f) concentrating the mixture from step (e) to provide a residue and partitioning the residue between water and a volatile organic solvent and concentrating the water phase to provide a residue, which is a mixture of 11 and 12.

In step (b), the tertiary amine may be selected from a wide variety of tertiary amines. Non-limiting illustrative examples of tertiary amines include pyridine, triethylamine, N,N′-diisopropylamine (DIPEA), and 4-dimethylaminopyridine (DMAP). The tertiary amine is preferably triethylamine.

In a preferred embodiment of this invention, the method for preparing a mixture of 11 and 12 comprises (a) cooling dichloromethane and 3-(2-fluoro-1-oxopropyl)-2(3H)-benzoxazolone to about 0° C.; (b) admixing to the mixture in step (a) 1.0 M solution of titanium (IV) in dichloromethane followed by triethylamine; (c) stirring the mixture from step (b) for about 1 h and adding a solution of D-glyceraldehyde, 1,2-acetonide in dichloromethane; (d) stirring the mixture from step (c) at about 0° C. for 30 minutes and quenching the mixture with 6% HCl; (e) extracting the mixture from step (d) with a volatile organic solvent and concentrating the solvent to provide an intermediate; (f) mixing the intermediate from step (e) with acetic acid and water and stirring the mixture at about 90° C. for about 3 h; and (g) concentrating the mixture from step (f) to provide a residue and partitioning the residue between water and tert-butyl methyl ether and concentrating the water phase to provide a residue, which contains 78% of 11 and 22% of 12.

The present invention also provides novel compounds 4 and 5 having the formulae set out below:

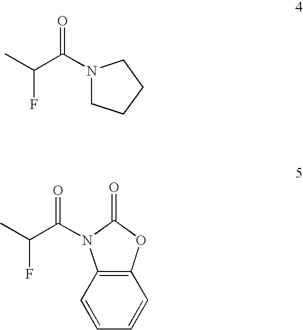

The compounds of the present invention can be prepared according to the examples set out below. The examples are presented for purposes of demonstrating, but not limiting, the preparation of the compounds and compositions of this invention.

### EXAMPLES

In accordance with the present invention, the following examples are provided to illustrate methods for preparing compounds (2R)-2-deoxy-2-fluoro-2-methyl-D-erythro-pentono-γ-lactone (11) and (2S)-2-deoxy-2-fluoro-2-methyl-D-erythro-pentono-γ-lactone (12), which are intermediates for preparing a key intermediate 3,5-di-O-acyl-2-fluoro-2-C-methyl-D-ribono-γ-lactone (B), for the preparation of 1-(2-deoxy-2-fluoro-2-C-methyl-β-D-ribofuranosyl)cytosine (A)

### Example 1

### Preparation of 4 from 2-Fluoropropionic acid (1) and Pyrrolidine (2)

To a solution of 4 g of 2-fluoropropionic acid (1) and a catalytic amount of dimethylformamide (DMF) in 50 mL of anhydrous toluene was slowly added 6.1 g of oxalyl chloride. The mixture was stirred at room temperature for 2 h and at 30° C. for 1 h, and then was cooled to −70° C. To the mixture was added 10 g of pyrrolidine (2). After the addition, the mixture was slowly warmed to ambient temperature. The mixture was washed consecutively with 5% HCl solution, 5% NaHCO₃ solution, and brine. The organic solution was dried over MgSO₄, filtered, and concentrated to give 2 g of 4. ¹H NMR (CDCl₃): δ=1.5 (dd, 3H, J=28, 7.0 Hz), 1.75-1.85 (m, 2H), 1.85-2.0 (m, 2H), 3.4-3.6 (m, 4H), 5.1 (dq, 1H, J=51, 7.0 Hz).

### Example 2

### Preparation of 5 from 2-Fluoropropionic acid (1) and 3

A solution of 5 g of 2-fluoropropionic acid (1), 5.6 g of 2-benzoxazolinone (3), and a catalytic amount of 4-dimethylaminopyridine (DMAP) in 40 mL of dichloromethane was cooled to 0° C. To this solution was added a solution of 11.2 g of N,N′-dicyclohexylcarbodiimide (DCC) in 20 mL of dichloromethane in one portion. Heavy precipitation formed immediately. The mixture was warmed slowly to ambient temperature and was stirred overnight. The solid was filtered off and the filtrate was concentrated to give a crude product. The crude product was purified with column chromatography (silica gel), eluting with dichloromethane to give 6.4 g of 5. ¹H NMR (CDCl₃): δ=1.74 (dd, 3H, J=23.2, 6.8 Hz), 6.10 (dq, 1H, J=48.4, 6.8 Hz), 7.22-7.36 (m, 3H), 8.08-8.14 (m, 1H).

### Example 3

### Preparation of 11 and 12 from 4 and D-Glyceraldehyde, 1,2-acetonide (6)

A dry, clean, 4-neck round bottom flask, equipped with a mechanical stirrer, a thermo couple, a nitrogen inlet, and an addition funnel, was charged with 5 mL of tetrahydrofuran (THF) and 2.3 mL of 1.8 M solution of lithium diisopropylamide in heptane/THF/ethylbenzene. The solution was cooled to −78° C. and to it was slowly charged a solution of 0.6 g of 4 in 3 mL of THF. The mixture was stirred for 30 minutes and a solution of 0.48 g of D-glyceraldehyde, 1,2-acetonide (6) in 3 mL of THF was slowly added. After the addition, the mixture was stirred at approximately −75° C. for 10 minutes and was quenched with a mixture of acetic acid and THF. The mixture was partitioned between dichloromethane and water. The organic layer was separated, dried over MgSO₄, filtered and concentrated to give crude intermediate 8, which was not isolated or characterized.

Crude intermediate 8 was mixed with 6 mL of acetic acid and 4 mL of water. The mixture was stirred at 90° C. overnight. The mixture was concentrated to dryness and the residue was partitioned between water and tert-butyl methyl ether (TBME). The aqueous phase was separated and concentrated to dryness to give 150 mg of a product that contained 63% of 11 and 37% of 12. ¹H NMR (DMSO-d⁶) for 11: δ=1.46 (d, 3H, J=24 Hz), 3.55 (dd, 1H, J=12.8, 4.4 Hz), 3.73-3.80 (m, 1H), 3.96 (dd, 1H, J=24, 8 Hz), 4.20-4.28 (m, 1H). ¹H NMR (DMSO-d⁶) for 12: δ=1.48 (d, 3H, J =24 Hz), 3.40-3.70 (m, 1H), 3.75-3.95 (m, 1H), 4.05-4.15 (m, 1H), 4.30-4.50 (m, 1H).

### Example 4

### Preparation of 11 and 12 from 7 and D-Glyceraldehyde, 1,2-acetonide (6)

A dry, clean, 4-neck round bottom flask, equipped with a mechanical stirrer, a thermo couple, a nitrogen inlet, and an addition funnel, was charged with 5 mL of THF and 2.3 mL of 1.8 M solution of lithium diisopropylamide in heptane/THF/ethylbenzene. The solution was cooled to −78° C. and to it was slowly charged a solution of 0.8 g of S-phenyl-2-fluoropropanethioate (7) in 5 mL of THF. The mixture was stirred for 1 h and a solution of 0.5 g of D-glyceraldehyde, 1,2-acetonide (6) in 5 mL THF was slowly added. After the addition, the mixture was stirred at approximately −75° C. for 10 minutes and was quenched with a mixture of acetic acid and THF. The mixture was partitioned between dichloromethane and water. The organic layer was separated, dried over MgSO₄, filtered and concentrated to give crude intermediate 9, which was not isolated or characterized.

Crude intermediate 9 was mixed with 6 mL of acetic acid and 4 mL of water. The mixture was stirred at 90° C. for 4 h. The mixture was concentrated to dryness and the residue was partitioned between water and TBME. The aqueous phase was separated and concentrated to dryness to give 940 mg of a product that contained 28% of 11 and 72% of 12.

### Example 5

### Preparation of 11 and 12 from 7, Chlorotriisopropoxytitanium (IV), and D-Glyceraldehyde, 1,2-acetonide (6)

A dry, clean, 4-neck round bottom flask, equipped with a mechanical stirrer, a thermo couple, a nitrogen inlet, and an addition funnel, was charged with 5 mL of THF and 2.3 mL of 1.8 M solution of lithium diisopropylamide in heptane/THF/ethylbenzene. The solution was cooled to −78° C. and to it was slowly charged a solution of 0.8 g of 7 in 5 mL of THF. The mixture was stirred for 15 minutes and 2.2 g of chlorotriisopropoxytitanium (IV) was added. The mixture was stirred for 1 h and a solution of 0.5 g of D-glyceraldehyde, 1,2-acetonide (6) in 5 mL THF was slowly added. After the addition, the mixture was stirred at approximately −75° C. for 10 minutes and was quenched with a mixture of acetic acid and THF. The mixture was partitioned between dichloromethane and water. The organic layer was separated, dried over MgSO₄, filtered and concentrated to give crude intermediate 9, which was not isolated or characterized.

Crude intermediate 9 was mixed with 6 mL of acetic acid and 4 mL of water. The mixture was stirred at 90° C. for 4 h. The mixture was concentrated to dryness and the residue was partitioned between water and TBME. The aqueous phase was separated and concentrated to dryness to give 940 mg of a product that contained 45% of 11 and 55% of 12.

### Example 6

### Preparation of 11 and 12 from 5 and D-Glyceraldehyde, 1,2-acetonide (6)

A dry, clean, 4-neck round bottom flask, equipped with a mechanical stirrer, a thermocouple, a nitrogen inlet, and an addition funnel, was charged with 10 mL of dichloromethane and 0.62 g of 5. The mixture was cooled to 0° C. and to it was added 3.5 mL of 1.0 M solution of titanium (IV) chloride in dichloromethane, followed by 0.36 g of triethylamine. The mixture was stirred for 1 h and a solution of 0.5 g of D-glyceraldehyde, 1,2-acetonide (6) in 5 mL dichloromethane was slowly added. After the addition, the mixture was stirred at approximately 0° C. for 30 minutes and was quenched with 20 mL of 6% HCl. The mixture was transferred to a separatory funnel. The aqueous phase was separated and extracted with dichloromethane. The combined organic solution was washed with saturated NaHCO₃ solution, dried over MgSO₄, filtered and concentrated to give a crude intermediate 10, which was not isolated or characterized.

Crude intermediate 10 was mixed with 6 mL of acetic acid and 4 mL of water. The mixture was stirred at 90° C. for 3 h. The mixture was concentrated to dryness and the residue was partitioned between water and TBME. The aqueous phase was separated and concentrated to dryness to give a product that contained 78% of 11 and 22% of 12.

While a number of embodiments of this invention have been represented, it is apparent that the basic construction can be altered to provide other embodiments that utilize the invention without departing from the spirit and scope of the invention. All such modifications and variations are intended to be included within the scope of the invention as defined in the appended claims rather than the specific embodiments that have been presented by way of example.

We claim:

1. A method for preparing compound 4 of the formula: 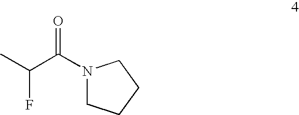
which comprises:
(a) converting 2-fluoropropionic acid to 2-fluoropropionyl chloride in a non-reactive solvent; and
(b) adding pyrrolidine to the mixture in step (a) to provide compound 4.

2. The method according to claim 1, wherein the reagent used is an acyl halide.

3. The method according to claim 1, wherein the non-reactive solvent is toluene.

4. The method according to claim 1, further comprising a catalytic amount of dimethylformamide in step (a).

5. The method according to claim 1, comprising:
(a) adding oxalyl chloride to a solution of 2-fluoropropionic acid and a catalytic amount of dimethylformamide in anhydrous toluene;
(b) stirring the mixture from step (a) at room temperature for about 2 h and at about 30° C. for about 1 h and then cooling the mixture to about −70° C.;
(c) adding pyrrolidine to the mixture in step (b) and warming the mixture to ambient temperature; and
(d) washing the mixture from step (c) with 5% HCl aqueous solution and 5% NaHCO₃ aqueous solution and concentrating the mixture to provide compound 4.

6. A method for preparing compound 5 of the formula: 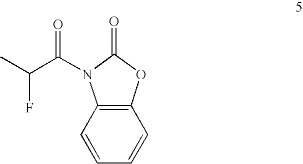
which comprises:
(a) adding a catalytic amount of a hypernucleophilic acylation catalyst to 2-fluoropropionic acid and 2-benzoxazolinone in a non-reactive solvent; and
(b) adding a dehydrating agent to the mixture in step (a) with stirring; and
(c) filtering the mixture in step (b) and concentrating the filtrate to provide compound 5.

7. The method according to claim 6, wherein the hypernucleophilic acylation catalyst is 4-dimethylaminopyridine.

8. The method according to claim 6, wherein the dehydrating agent is a carbodiimide selected from the group consisting of N,N′-dicyclohexylcarbodiimide, N,N′-diisopropylcarbodiimide, and 1-ethyl-3-(3-dimethylaminopropyl)carbodiimide hydrochloride.

9. The method according to claim 8, wherein the dehydrating agent is N,N′-dicyclohexylcarbodiimide.

10. The method according to claim 6, comprising:
(a) adding 2-fluoropropionic acid, 2-benzoxazolinone, and a catalytic amount of 4-dimethylaminopyridine in dichloromethane and cooling the mixture to about 0° C.;
(b) adding N,N′-dicyclohexylcarbodiimide in dichloromethane to the mixture in step (a);
(c) warming the mixture from step (b) to ambient temperature and stirring the mixture overnight; and
(d) filtering the mixture from step (c) and concentrating the filtrate to provide compound 5.

11. A method for preparing a mixture of compounds of the formula 11 and 12: 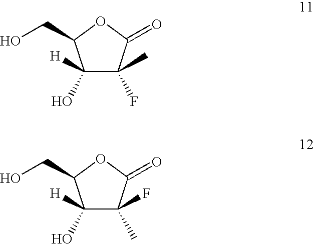
which comprises:
(a) admixing a non-nucleophilic base and 1-(2-fluoro-1-oxopropyl)pyrrolidine in a non-reactive solvent;
(b) admixing D-glyceraldehyde, 1,2-acetonide to the mixture from step (a);
(c) neutralizing the mixture from step (b) and partitioning the resulting mixture between a volatile organic solvent and water and concentrating the volatile organic solution to provide an intermediate;
(d) hydrolyzing the intermediate from step (c); and
(e) concentrating the mixture from step (d) to provide a residue and partitioning the residue between water and a volatile organic solvent and concentrating the water phase to provide a residue, which is a mixture of 11 and 12.

12. The method according to claim 11, wherein the non-nucleophilic base is selected from the group consisting of lithium diisopropylamide, lithium 2,2,6,6-tetramethylpiperidine, and lithium hexamethyldisilazide.

13. The method according to claim 11, comprising:
(a) admixing tetrahydrofuran and 1.8 M solution of lithium diisopropylamide in heptane/tetrahydrofuran/ethylbenzene;
(b) cooling the mixture from step (a) to about −78° C. and admixing 1-(2-fluoro-1-oxopropyl)pyrrolidine in tetrahydrofuran;
(c) stirring the mixture from step (b) for about 30 minutes and adding a solution of D-glyceraldehyde, 1,2-acetonide in tetrahydrofuran;
(d) stirring the mixture from step (c) at about −75° C. for about 10 minutes and quenching the mixture with a solution of acetic acid in tetrahydrofuran;
(e) partitioning the mixture from step (d) between dichloromethane and water and concentrating the dichloromethane phase to provide an intermediate;
(f) mixing the intermediate from step (e) with acetic acid and water and stirring the mixture at about 90° C. overnight; and
(g) concentrating the mixture from step (f) to provide a residue and partitioning the residue between water and tert-butyl methyl ether and concentrating the water phase to provide a residue, which contains 63% of 11 and 37% of 12.

14. A method for preparing a mixture of compounds of the formula 11 and 12: 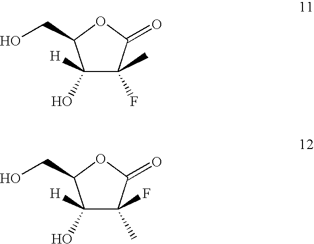
which comprises:
(a) admixing a non-nucleophilic base and S-phenyl-2-fluoropropanethioate in a non-reactive solvent;
(b) admixing D-glyceraldehyde, 1,2-acetonide to the mixture from step (a);
(c) neutralizing the mixture from step (b) and partitioning the resulting mixture between a volatile organic solvent and water and concentrating the volatile organic solution to provide an intermediate;
(d) hydrolyzing the intermediate from step (c); and
(e) concentrating the mixture from step (d) to provide a residue and partitioning the residue between water and a volatile organic solvent and concentrating the water phase to provide a residue, which is a mixture of 11 and 12.

15. The method according to claim 14, wherein the non-nucleophilic base is selected from the group consisting of lithium diisopropylamide, lithium 2,2,6,6-tetramethylpiperidine, and lithium hexamethyldisilazide.

16. The method according to claim 14, comprising:
(a) admixing tetrahydrofuran and 1.8 M solution of lithium diisopropylamide in heptane/tetrahydrofuran/ethylbenzene;
(b) cooling the mixture from step (a) to about −78° C. and admixing S-phenyl-2-fluoropropanethioate in tetrahydrofuran;
(c) stirring the mixture from step (b) for about 30 minutes and adding a solution of D-glyceraldehyde, 1,2-acetonide in tetrahydrofuran;
(d) stirring the mixture from step (c) at about −75° C. for about 10 minutes and quenching the mixture with a solution of acetic acid in tetrahydrofuran;
(e) partitioning the mixture from step (d) between dichloromethane and water and concentrating the dichloromethane phase to provide an intermediate;
(f) mixing the intermediate from step (e) with acetic acid and water and stirring the mixture at about 90° C. overnight; and
(g) concentrating the mixture from step (f) to provide a residue and partitioning the residue between water and tert-butyl methyl ether and concentrating the water phase to provide a residue, which contains 45% of 11 and 55% of 12.

17. A method for preparing a mixture of compounds of the formula 11 and 12: 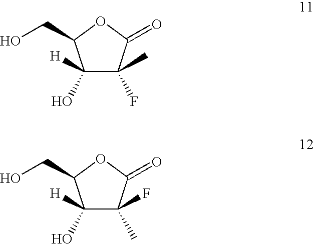
which comprises:
(a) admixing a non-nucleophilic base, S-phenyl-2-fluoropropanethioate, and chlorotriisopropoxytitanium (IV) in a non-reactive solvent;
(b) admixing D-glyceraldehyde, 1,2-acetonide to the mixture from step (a);
(c) neutralizing the mixture from step (b) and partitioning the resulting mixture between a volatile organic solvent and water and concentrating the volatile organic solution to provide an intermediate;
(d) hydrolyzing the intermediate from step (c); and
(e) concentrating the mixture from step (d) to provide a residue and partitioning the residue between water and a volatile organic solvent and concentrating the water phase to provide a residue, which is a mixture of 11 and 12.

18. The method according to claim 17, wherein the non-nucleophilic base is selected from the group consisting of lithium diisopropylamide, lithium 2,2,6,6-tetramethylpiperidine, and lithium hexamethyldisilazide.

19. The method according to claim 17, comprising:
(a) admixing tetrahydrofuran and a 1.8 M solution of lithium diisopropylamide in heptane/tetrahydrofuran/ethylbenzene;
(b) cooling the mixture from step (a) to about −78° C. and then admixing S-phenyl-2-fluoropropanethioate in tetrahydrofuran;
(c) stirring the mixture from step (b) for about 1 h and then admixing chlorotriisopropoxytitanium (IV);
(d) stirring the mixture from step (c) for about 1 h and adding a solution of D-glyceraldehyde, 1,2-acetonide in tetrahydrofuran;
(e) stirring the mixture from step (d) at about −75° C. for about 10 minutes and quenching the mixture with a solution of acetic acid in tetrahydrofuran;
(f) partitioning the mixture from step (e) between dichloromethane and water and concentrating the dichloromethane phase to provide an intermediate;
(g) mixing the intermediate from step (f) with acetic acid and water and stirring the mixture at about 90° C. overnight; and
(h) concentrating the mixture from step (g) to provide a residue and partitioning the residue between water and tert-butyl methyl ether and concentrating the water phase to provide a residue which contains 28% of 11 and 72% of 12.

20. A method for preparing a mixture of compounds of the formula 11 and 12: 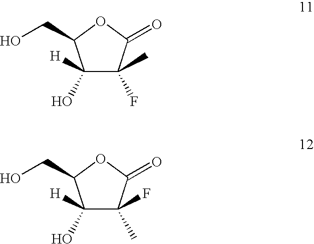
which comprises:
(a) admixing 3-(2-fluoro-1-oxopropyl)-2(3H)-benzoxazolone to a non-reactive solvent;
(b) admixing titanium (IV) chloride and a tertiary amine base to the mixture from step (a);
(c) admixing D-glyceraldehyde, 1,2-acetonide to the mixture from step (b);
(d) neutralizing the mixture from step (c) and partitioning the resulting mixture between a volatile organic solvent and water and concentrating the volatile organic solution to provide an intermediate;
(e) hydrolyzing the intermediate from step (d); and
(f) concentrating the mixture from step (e) to provide a residue and partitioning the residue between water and a volatile organic solvent and concentrating the water phase to provide a residue, which is a mixture of 11 and 12.

21. The method according to claim 20, comprising:
(a) cooling dichloromethane and 3-(2-fluoro-1-oxopropyl)-2(3H)-benzoxazolone to about 0° C.;
(b) admixing to the mixture in step (a) 1.0 M solution of titanium (IV) in dichloromethane followed by triethylamine;
(c) stirring the mixture from step (b) for about 1 h and adding a solution of D-glyceraldehyde, 1,2-acetonide in dichloromethane;
(d) stirring the mixture from step (c) at about 0° C. for 30 minutes and quenching the mixture with 6% HCl;
(e) extracting the mixture from step (d) with a volatile organic solvent and concentrating the solvent to provide an intermediate;
(f) mixing the intermediate from step (e) with acetic acid and water and stirring the mixture at about 90° C. for about 3 h; and
(g) concentrating the mixture from step (f) to provide a residue and partitioning the residue between water and tert-butyl methyl ether and concentrating the water phase to provide a residue, which contains 78% of 11 and 22% of 12.

22. A compound 4 having the formula set out below:

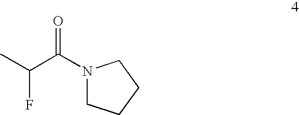

23. A compound 5 having the formula set out below: [chemical structure]
